(19) United States
(12) Patent Application Publication
(10) Pub. No.: US 2023/0370529 A1
(43) Pub. Date: Nov. 16, 2023

(54) INFORMATION PROCESSING APPARATUS, INFORMATION PROCESSING METHOD, AND PROGRAM

(71) Applicants: NEC CORPORATION, Tokyo (JP); MACROMILL, INC., Tokyo (JP)

(72) Inventors: Kei TAKEMURA, Tokyo (JP); Junko OCHIAI, Tokyo (JP); Takuya KOMATSU, Tokyo (JP)

(73) Assignees: NEC CORPORATION, Tokyo (JP); MACROMILL, INC., Tokyo (JP)

(21) Appl. No.: 18/197,369

(22) Filed: May 15, 2023

(30) Foreign Application Priority Data
May 16, 2022 (JP) 2022-079977

(51) Int. Cl. H04L 67/50; G09B 7/06; A61B 5/16

(52) U.S. Cl. CPC H04L 67/535; A61B 5/165; G09B 7/06

(57) ABSTRACT

Provided is an information processing apparatus including: an acquisition unit that acquires information indicating a usage state of each of at least one or more users with respect to a specific function, and information indicating a psychological degree being at least one of a recognition degree and a satisfaction degree of each user with respect to the specific function; an estimation unit that estimates, based on the information acquired by the acquisition unit, a user’s usage state with respect to the specific function in a case where a psychological degree of a user with respect to the specific function is improved; and an output unit that outputs information based on an estimation result by the estimation unit.

### INCORPORATION BY REFERENCE

This application is based upon and claims the benefit of priority from Japanese patent application No. 2022-079977, filed on May 16, 2022, the disclosure of which is incorporated herein in its entirety by reference.

### TECHNICAL FIELD

The present disclosure relates to an information processing apparatus, an information processing method, and a program.

### BACKGROUND ART

Various techniques for retention (retention of existing customers) are known. Regarding such techniques, Patent Literature 1 discloses a technique of calculating, as a usage fee for a predetermined period, an amount acquired by reducing a fixed fee for each predetermined period according to a good-customer index.

[Patent Literature 1] Japanese Unexamined Patent Application Publication No. 2021-099716

However, in the technique described in Patent Literature 1, there is a problem that, for example, retention or the like may not be performed in some cases.

### SUMMARY

An example object of the present disclosure is to provide a technique of being able to appropriately acquire a new customer or retain an existing customer in view of the above-described problem.

In a first aspect of the present disclosure, an information processing apparatus includes: an acquisition unit configured to acquire information indicating a usage state of each of at least one or more users relating to a specific function, and information indicating a psychological degree being at least one of a recognition degree and a satisfaction degree of each user with respect to the specific function; an estimation unit configured to estimate, based on the information acquired by the acquisition unit, a user’s usage state relating to the specific function in a case where the psychological degree of the user with respect to the specific function is improved; and an output unit configured to output information based on an estimation result by the estimation unit.

In a second aspect of the present disclosure, an information processing method includes: acquiring information indicating a usage state of each of at least one or more users with respect to a specific function, and information indicating a psychological degree being at least one of a recognition degree and a satisfaction degree of each user with respect to the specific function; estimating, based on the acquired information, a user’s usage state with respect to the specific function in a case where the psychological degree of the user with respect to the specific function is improved; and outputting information based on an estimation result.

Further, in a third aspect of the present disclosure, a program causes a computer to execute processing of: acquiring information indicating a usage state of each of at least one or more users with respect to a specific function, and information indicating a psychological degree being at least one of a recognition degree and a satisfaction degree of each user with respect to the specific function; estimating, based on the acquired information, a user’s usage state with respect to the specific function in a case where the psychological degree of the user with respect to the specific function is improved; and outputting information based on an estimation result.

An example object of the invention is to appropriately acquire a new customer or retain an existing customer.

### BRIEF DESCRIPTION OF DRAWINGS

The above and other aspects, features, and advantages of the present disclosure will become more apparent from the following description of certain example embodiments when taken in conjunction with the accompanying drawings, in which:

FIG. 1 is a diagram illustrating one example of a configuration of an information processing apparatus according to embodiments;

FIG. 2 is a diagram illustrating one example of a configuration of an information processing system according to the embodiments;

FIG. 3 is a diagram illustrating a hardware configuration example of the information processing apparatus according to the embodiments;

FIG. 4 is a flowchart illustrating one example of processing performed by the information processing apparatus according to the embodiments; and

FIG. 5 is a diagram illustrating one example of information stored in a user database according to the embodiments.

### EXAMPLE EMBODIMENTS

The principles of the present disclosure are described with reference to several exemplary embodiments. It should be understood that these embodiments are set forth for the purpose of illustration of examples only and do not suggest limitations on the scope of the present disclosure, and the embodiments help those skilled in the art understand and practice the present disclosure. The disclosure described in the present specification may be implemented in various methods other than those described below.

In the following description and claims, unless otherwise defined, all technical and scientific terms used herein have the same meaning as commonly understood by those of ordinary skill in the art to which this disclosure belongs.

Hereinafter, embodiments of the present disclosure will be described with reference to the drawings.

### First Embodiment

### <Configuration>

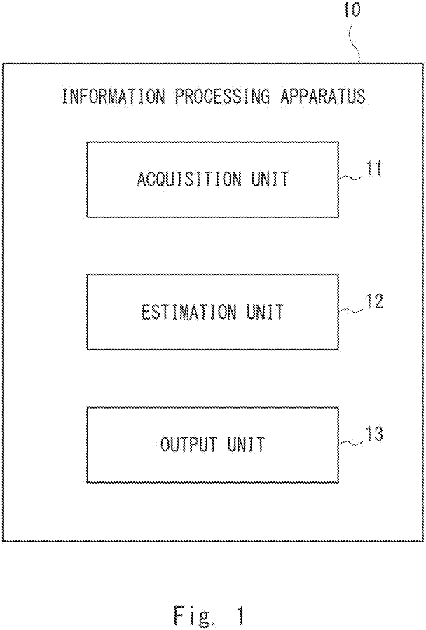

A configuration of an information processing apparatus 10 according to an embodiment will be described with reference to FIG. 1. FIG. 1 is a diagram illustrating one example of the configuration of the information processing apparatus 10 according to the embodiment. The information processing apparatus 10 includes an acquisition unit 11, an estimation unit 12, and an output unit 13. These units may be achieved by cooperation of one or more programs installed in the information processing apparatus 10 and hardware such as a processor 101 and a memory 102 of the information processing apparatus 10.

The acquisition unit 11 acquires, for example, a usage state of each of at least one user relating to a specific function and information indicating a psychological degree that is at least one of the recognition degree and the satisfaction degree of each user with respect to the specific function. The estimation unit 12 estimates, for example, based on the information acquired by the acquisition unit 11, a user’s usage state relating to a specific function in a case where a psychological degree of the user with respect to the specific function is improved. The output unit 13 outputs, for example, information based on the estimation result by the estimation unit 12.

A function according to the present disclosure refers to elements in a system provided to a customer. For example, a system that performs information retrieval, based on a query received from a user, will be described. In this case, it is a function to receive information relating to an extension and date from the user and narrow down the search result. Further, it is also a function to propose a similar query, based on a query received from a user. Note that, in the present embodiment, a function is not limited to an element in the system, and may be interpreted as a product or a service provided to a customer.

### Second Embodiment

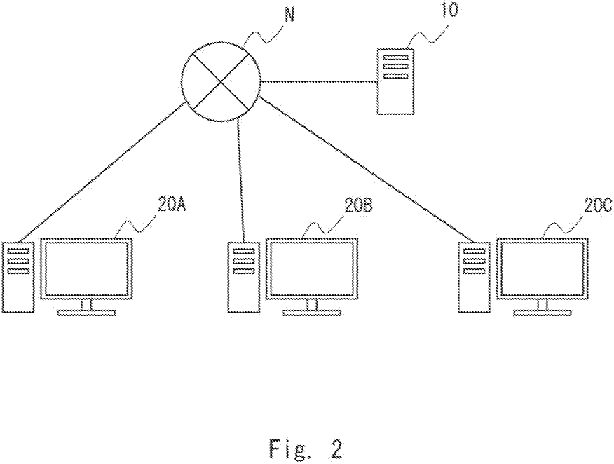

Next, a configuration of an information processing system 1 according to the embodiment will be described with reference to FIG. 2.

### <System Configuration>

FIG. 2 is a diagram illustrating one example of a configuration of the information processing system 1 according to the embodiment. In the example of FIG. 2, the information processing system 1 includes an information processing apparatus 10. Further, the information processing system 1 includes a user terminal 20A, a user terminal 20B, and a user terminal 20C (hereinafter, simply referred to as “a user terminal 20” when there is no need to distinguish between the user terminals). In the example of FIG. 2, the information processing apparatus 10 and the user terminal 20 are connected so as to be able to communicate with each other via a network N. Note that the numbers of the information processing apparatuses 10 and the user terminals 20 are not limited to the example of FIG. 2.

Examples of the network N include, for example, the Internet, a mobile communication system, a wireless local area network (LAN), a LAN, a bus, and the like. Examples of mobile communication systems include, for example, a fifth-generation mobile communication system (5G), a sixth-generation mobile communication system (6G, Beyond 5G), a fourth-generation mobile communication system (4G), a third-generation mobile communication system (3G), and the like.

The information processing apparatus 10 may be, for example, an apparatus such as a server, a cloud, a personal computer, a smartphone, or the like. The information processing apparatus 10 performs a simulation for retaining an existing customer and acquiring a new customer, based on, for example, a usage history of various functions and the like by a user and an answer result of a questionnaire returned from a user.

The user terminal 20 may be, for example, a terminal such as a personal computer, a smartphone, a tablet, a car navigation device, a gaming machine, or the like owned by a user. The user terminal 20 performs, for example, processing using various functions and the like provided by the information processing apparatus 10 or another apparatus in response to an operation or the like from the user.

### <Hardware Configuration>

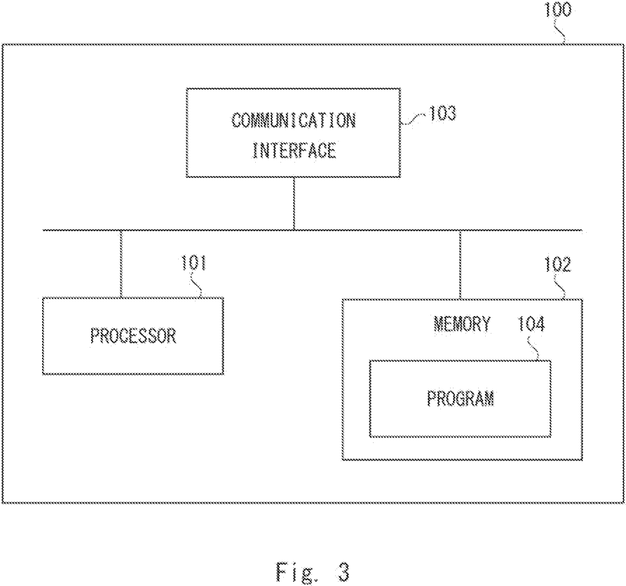

FIG. 3 is a diagram illustrating a hardware configuration example of the information processing apparatus 10 according to the embodiment. In the example of FIG. 3, the information processing apparatus 10 (computer 100) includes a processor 101, a memory 102, and a communication interface 103. These units may be connected by a bus or the like. The memory 102 stores at least a part of a program 104. The communication interface 103 includes an interface necessary for communication with other network elements.

When the program 104 is executed by the cooperation of the processor 101 and the memory 102, the computer 100 executes at least a part of processing of the embodiment according to the present disclosure. The memory 102 may be of any type suitable for a local technology network. The memory 102 may be, as a non-limiting example, a non-transitory computer readable storage medium. The memory 102 may also be implemented by using any suitable data storage technology, such as a semiconductor-based memory device, magnetic memory device or system, optical memory device or system, fixed memory, removable memory, or the like. Although only one memory 102 is illustrated in the computer 100, there may be several physically different memory modules included in the computer 100. The processor 101 may be of any type. The processor 101 may include one or more of a general-purpose computer, a dedicated computer, a microprocessor, a digital-signal processor (DSP), and, as a non-limiting example, a processor which is based on a multi-core processor architecture. The computer 100 may have a plurality of processors, such as an application specific integrated circuit chip, which is temporally dependent on a clock that synchronizes the main processor.

Embodiments according to the present disclosure may be implemented in hardware or dedicated circuit, software, logic, or any combination thereof. Some aspects may be implemented in hardware, while other aspects may be implemented in firmware or software that may be executed by a controller, a microprocessor, or other computing devices.

The present disclosure also provides at least one computer program product tangibly stored on a non-transitory computer readable storage medium. The computer program product includes computer-executable instructions, such as instructions contained in a program module, and is executed by a device on a real processor or virtual processor of interest to execute the processes or methods of the present disclosure. Program modules include routines, programs, libraries, objects, classes, components, data structures, and the like, that perform particular tasks or implement particular abstract data types. The function of the program modules may be combined or split between the program modules as desired in various embodiments. The machine-executable instructions of the program modules may be executed in a local or distributed device. In a distributed device, program modules may be located on both local and remote storage media.

A program code for executing the methods according to the present disclosure may be written in any combination of one or more programming languages. The program codes are provided to a processor or controller of a general-purpose computer, a dedicated computer, or other programmable data processing devices. When the program code is executed by a processor or a controller, functions/operations in the flowcharts and/or implementing block diagrams are performed. The program code is executed entirely on a machine or partly on a machine, or executed, as a stand-alone software package, partly on a machine, partly on a remote machine, or entirely on a remote machine or a server.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g., magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

### <Processing>

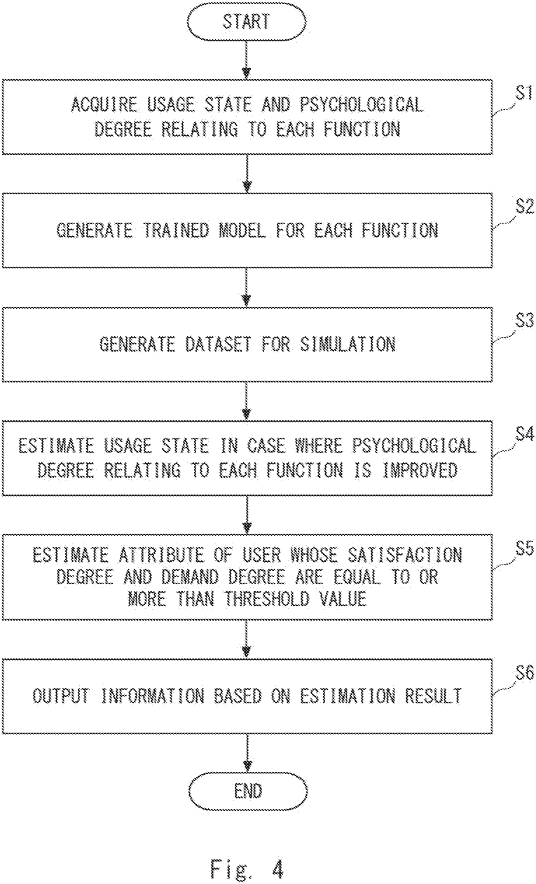

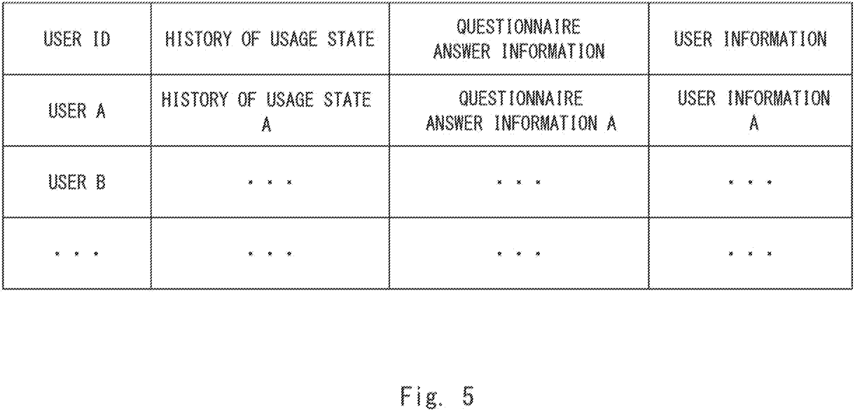

Next, one example of processing of the information processing apparatus 10 according to the embodiment will be described with reference to FIGS. 4 and 5. FIG. 4 is a flowchart illustrating one example of processing performed by the information processing apparatus 10 according to the embodiment. FIG. 5 is a diagram illustrating one example of information stored in a user database (DB) 501 according to the embodiment. Note that, the processing of FIG. 4 may be executed at a timing instructed by, for example, an operator (administrator) or the like of the information processing apparatus 10.

In step S1, the acquisition unit 11 acquires, from the user DB 501, information indicating a usage state of each user relating to each function and information indicating a psychological degree that is at least one of a recognition degree and a satisfaction degree of each user with respect to each function. Note that the user DB 501 may be recorded in a storage device inside the information processing apparatus 10 or may be recorded in a storage device outside the information processing apparatus 10. Information in the user DB 501 may be registered in advance by an administrator or the like.

In the example of FIG. 5, in the user DB 501, a history of a usage state, questionnaire answer information, and user information are recorded in association with a user ID. The user ID is identification information of a user of the user terminal 20.

The history of the usage state is a history of a user’s usage state of a specific function or service. The history of the usage state may include information indicating at least one of a login frequency to a specific service, a usage frequency for each function, and a billing status to a specific service.

The questionnaire answer information is information based on an answer to a questionnaire (question) about each function from a user. The questionnaire answer information may include information indicating a recognition degree, a satisfaction degree, and a demand degree for each function. The recognition degree is a degree to which the user knows the function. The satisfaction degree is the degree to which the user is satisfied with the function. The demand degree is a degree to which the user desires to use the function. Each of the recognition degree, the satisfaction degree, and the demand degree may be, for example, a value answered in a predetermined grade (for example, five grades) in response to questions such as “Do you know this function?”, “Are you satisfied with this function?”, “Do you want to use this function?” or the like. In this case, for example, the choices and values of the answers to the questionnaire regarding the recognition degree may be “Do not know at all” being [0], “Do not know much” being “1”, “Know a little” being “3”, “Know to some degree” being “4”, and “Know well” being “5”, or the like.

The user information is information relating to a user. The user information may include, for example, information indicating an attribute of the user. The attribute of the user may include, for example, gender, age, job type, and the like.

Subsequently, the estimation unit 12 generates a trained model for each function by performing supervised learning for each function, by using the information of the user DB 501 which has been acquired by the acquisition unit 11 as learning data (training data) (step S2). The estimation unit 12 may use, for example, a neural network, a regression analysis, or the like as a method of supervised learning.

Herein, for example, the estimation unit 12 may use any one of the items (for example, a login frequency to a specific service, a usage frequency of a function, and a billing state to a specific service) included in the usage state as a correct answer label (target variable). Then, for example, the estimation unit 12 may use, among the items included in the information of the user DB 501, an item other than the item set as the correct answer label as an explanatory variable.

Further, for example, the estimation unit 12 may use an item determined based on the usage state as a correct answer label, and use some or all of the items included in the information of the user DB 501 as the explanatory variable. In this case, whether a user is a loyal user or not may be used as the correct answer label. In this case, for example, the estimation unit 12 may determine that a user whose login frequency to a specific service is equal to or greater than a threshold value is a loyal user. Further, for example, the estimation unit 12 may determine that a user whose usage frequency for each function is equal to or greater than a threshold value is a loyal user. Further, for example, the estimation unit 12 may determine that a user whose billing state (for example, a billing amount grade or a billing amount) for a specific service is equal to or greater than a threshold value is a loyal user.

Subsequently, the estimation unit 12 generates a dataset for simulation, based on the information of the user DB 501 acquired by the acquisition unit 11 (step S3). Herein, the estimation unit 12 may generate, as a dataset for simulation, a dataset acquired by setting (changing) the value of the psychological degree of a user whose value of the psychological degree is less than the specified value to the specified value, the psychological degree being at least one of a recognition degree and a satisfaction degree included in the questionnaire answer information of each user recorded in the user DB 501. Note that the specified value may be set in advance or may be set by an operation of an administrator or the like. For example, when the specified value is “4”, a dataset in which the psychological degree of a user being less than “4” (i.e., “1”, “2”, or “3”) is set to “4” is generated.

In the case where any one of the items (for example, the login frequency to a specific service, the usage frequency of a function, and the billing status to a specific service) included in the usage state is used as the correct answer label, the estimation unit 12 may assume, for example, that the item of the correct answer label has no data (NULL).

Further, in a case where the item determined based on the usage state is used as the correct answer label, the estimation unit 12 may compare the correct answer label with the information recorded in the user DB 501 in FIG. 5, and may set a dataset and a dataset for simulation which differ only in the value of the psychological degree included in the questionnaire answer information.

Subsequently, the estimation unit 12 estimates (infers) a user’s usage state in a case where the psychological degree of the user for each function is improved, based on the dataset for simulation generated in step S3 and the trained model generated in step S2 (step S4). Accordingly, for example, in a case where the psychological degree of the user whose psychological degree being less than the specified value is improved to the specified value, the value of the item included in the usage state, the value of the item determined based on the usage state, and the like may be estimated. Therefore, for example, it becomes possible to estimate the effect of acquiring new customers or retaining existing customers by measures such as improving the recognition degree by advertising each function or improving the satisfaction degree by additional development and the like.

Subsequently, the estimation unit 12 estimates an attribute of the user whose satisfaction degree and/or degree are equal to or higher than a threshold value for a function among the plurality of functions (hereinafter, also appropriately referred to as a “target function”) in which the degree of change in the user’s usage state, in the case where the psychological degree of the user with respect to the function is improved, is equal to or higher than the threshold value (step S5). Herein, the estimation unit 12 may estimate the attribute of the user only for the target function.

The estimation unit 12 may estimate an attribute of a user whose satisfaction degree and/or demand degree is equal to or higher than a threshold value by using a known machine learning method such as, for example, a support vector machine (SVM), a multi-layer neural network, a principal component analysis, or clustering (e.g., k-means).

Subsequently, the output unit 13 outputs information based on the estimation result of the estimation unit 12 in at least one of steps S4 and S5 (step S6). Herein, the output unit 13 may notify of the information on the target function to a user, among the users having the attribute estimated by the estimation unit 12 in step S5, who has a recognition degree for the target function being less than the threshold value. Accordingly, it is possible to appeal the target function to, for example, a user of an attribute that is considered to have a high satisfaction degree or a high demand degree for the target function, and who has a low recognition degree for the target function. Note that, the output unit 13 may notify of the information of the target function via, for example, an e-mail, a pop-up screen at the time of login or the like, a privilege (for example, a discount coupon) for reducing the usage fee of the target function, or the like.

Further, for example, the output unit 13 may display, on a screen, the degree of change in the user’s usage state in a case where the psychological degree of the user for each function is improved, based on the information estimated by the estimation unit 12 in step S4. In this case, for example, the output unit 13 may display, for each function, a graph (for example, a histogram) of a value of the original psychological degree (psychological degree recorded in the user DB 501) and a value of the psychological degree after the improvement (psychological degree set in step S3), wherein the horizontal axis denotes the probability that each user becomes a loyal user and the vertical axis denotes the number of users. Further, the output unit 13 may display, for example, a list of the names of the respective functions and the values of the degree of change in the usage state in descending order of the degree of change in the usage state of the user (for example, the increase rate of the loyal user) in the case where the psychological degree of the user is improved.

### <Modification>

The information processing apparatus 10 may be, but is not limited to, an apparatus included in one housing. Each unit of the information processing apparatus 10 may be achieved by, for example, cloud computing configured by one or more computers. Further, the information processing apparatus 10 and the user terminal 20 may be housed in the same housing and configured as a single information processing apparatus. Further, the user terminal 20 may be configured to execute at least a part of processing of each functional unit of the information processing apparatus 10. Such an information processing apparatus 10 is also included in an example of “information processing apparatus” of the present disclosure.

Each of the above-described embodiments can be combined as desirable by one of ordinary skill in the art.

While the disclosure has been particularly shown and described with reference to embodiments thereof, the disclosure is not limited to these embodiments. It will be understood by those of ordinary skill in the art that various changes in form and details may be made therein without departing from the spirit and scope of the present disclosure as defined by the claims.

The whole or part of the exemplary embodiments described above can be described as, but not limited to, the following supplementary notes.

### (Supplementary Note 1)

An information processing apparatus including:
an acquisition unit configured to acquire information indicating a usage state of each of at least one or more users with respect to a specific function, and information indicating a psychological degree being at least one of a recognition degree and a satisfaction degree of each user with respect to the specific function;
an estimation unit configured to estimate, based on the information acquired by the acquisition unit, a user’s usage state with respect to the specific function in a case where a psychological degree of a user with respect to the specific function is improved; and
an output unit configured to output information based on an estimation result by the estimation unit.

### (Supplementary Note 2)

The information processing apparatus according to Supplementary note 1, wherein the estimation unit estimates a user’s usage state with respect to the specific function in a case where a psychological degree of a user, whose psychological degree for the specific function is less than a specified value among each of the users, becomes the specified value.

### (Supplementary Note 3)

The information processing apparatus according to Supplementary note 1 or 2, wherein, when a degree of change in a user’s usage state with respect to the specific function in a case where a psychological degree of the user with respect to the specific function is improved is equal to or greater than a threshold value, the estimation unit estimates an attribute of a user who has at least one of a satisfaction degree for the specific function according to a questionnaire and a demand degree for the specific function according to a questionnaire being equal to or greater than the threshold value.

### (Supplementary Note 4)

The information processing apparatus according to Supplementary note 3, wherein the output unit notifies of information on the specific function to a user, among users having an attribute estimated by the estimation unit, who has at least one of a usage frequency of the specific function and a satisfaction degree for the specific function according to a questionnaire being less than a threshold value.

### (Supplementary Note 5)

The information processing apparatus according to Supplementary note 1 or 2, wherein a usage state of each of the users with respect to the specific function includes information indicating at least one of a login frequency to a service providing the specific function, a usage frequency of the specific function, and a billing state to a service providing the specific function.

### (Supplementary Note 6)

The information processing apparatus according to Supplementary note 1 or 2, wherein information indicating a psychological degree of each of the users with respect to the specific function is information based on an answer to a questionnaire returned from each of the users.

### (Supplementary Note 7)

An information processing method including:
acquiring information indicating a usage state of each of at least one or more users with respect to a specific function, and information indicating a psychological degree being at least one of a recognition degree and a satisfaction degree of each user with respect to the specific function;
estimating, based on the acquired information, a user’s usage state with respect to the specific function in a case where a psychological degree of a user with respect to the specific function is improved; and
outputting information based on an estimation result.

### (Supplementary Note 8)

A program causing a computer to execute processing of:
acquiring information indicating a usage state of each of at least one or more users with respect to a specific function, and information indicating a psychological degree being at least one of a recognition degree and a satisfaction degree of each user with respect to the specific function;
estimating, based on the acquired information, a user’s usage state with respect to the specific function in a case where a psychological degree of a user with respect to the specific function is improved; and
outputting information based on an estimation result.

What is claimed is:

1. An information processing apparatus comprising:
an acquisition unit configured to acquire information indicating a usage state of each of at least one or more users with respect to a specific function, and information indicating a psychological degree being at least one of a recognition degree and a satisfaction degree of each user with respect to the specific function;
an estimation unit configured to estimate, based on the information acquired by the acquisition unit, a user’s usage state with respect to the specific function in a case where a psychological degree of a user with respect to the specific function is improved; and
an output unit configured to output information based on an estimation result by the estimation unit.

2. The information processing apparatus according to claim 1, wherein the estimation unit estimates a user’s usage state with respect to the specific function in a case where a psychological degree of a user, whose psychological degree for the specific function is less than a specified value among each of the users, becomes the specified value.

3. The information processing apparatus according to claim 1, wherein, when a degree of change in a user’s usage state with respect to the specific function in a case where a psychological degree of the user with respect to the specific function is improved is equal to or greater than a threshold value, the estimation unit estimates an attribute of a user who has at least one of a satisfaction degree for the specific function according to a questionnaire and a demand degree for the specific function according to a questionnaire being equal to or greater than a threshold value.

4. The information processing apparatus according to claim 3, wherein the output unit notifies of information on the specific function to a user, among users having an attribute estimated by the estimation unit, who has at least one of a usage frequency of the specific function and a satisfaction degree for the specific function according to a questionnaire being less than a threshold value.

5. The information processing apparatus according to claim 1, wherein a usage state of each of the users with respect to the specific function includes information indicating at least one of a login frequency to a service providing the specific function, a usage frequency of the specific function, and a billing state to a service providing the specific function.

6. The information processing apparatus according to claim 1, wherein information indicating a psychological degree of each of the users with respect to the specific function is information based on an answer to a questionnaire returned from each of the users.

7. An information processing method comprising:
acquiring information indicating a usage state of at least one or more users with respect to a specific function, and information indicating a psychological degree being at least one of a recognition degree and a satisfaction degree of each user with respect to the specific function;
estimating, based on the acquired information, a user’s usage state with respect to the specific function in a case where a psychological degree of a user with respect to the specific function is improved; and
outputting information based on an estimation result.

8. A non-transitory computer readable medium storing a program causing a computer to execute processing of:
acquiring information indicating a usage state of each of at least one or more users with respect to a specific function, and information indicating a psychological degree being at least one of a recognition degree and a satisfaction degree of each user with respect to the specific function;
estimating, based on the acquired information, a user’s usage state with respect to the specific function in a case where a psychological degree of a user with respect to the specific function is improved; and
outputting information based on an estimation result.
